(19) United States
(12) Patent Application Publication
(10) Pub. No.: US 2015/0379979 A1
(43) Pub. Date: Dec. 31, 2015

(54) METHODS AND COMPOSITIONS FOR REVERSIBLY REDUCING THE AURAL SENSITIVITY OF HUMANS AND ANIMALS

(71) Applicant: David CHAMBERLAIN, Guernsey (GB)

(72) Inventor: David CHAMBERLAIN, Guernsey (GB)

(21) Appl. No.: 14/754,684

(22) Filed: Jun. 29, 2015

Related U.S. Application Data
(63) Continuation of application No. 13/643,295, filed on Mar. 5, 2013, now abandoned.

(30) Foreign Application Priority Data
Apr. 27, 2010 (GB) 1007011.8

(51) Int. Cl. G10K 11/162; A61L 31/14; A01K 15/02

(52) U.S. Cl. CPC G10K 11/162; A01K 15/02; A61L 31/14; A61F 11/08

(57) ABSTRACT

The invention relates to an acoustic dampening fluid composition for reversibly, reducing the sensitivity of humans and animals to sounds such as fireworks to alleviate phobias in animals and to protect the hearing of humans exposed to sounds which would otherwise damage their hearing.

The invention relates to acoustic dampening fluid compositions and methods of using such compositions for reversibly, reducing the sensitivity of humans and animals to sounds such as fireworks to alleviate phobias in animals and to protect the hearing of humans exposed to sounds which would otherwise damage their hearing.

Conventional human ear defenders have the following disadvantages they are obtrusive, uncomfortable, easily dislodged cumbersome and unsightly to wear; whilst, ear plugs are potentially dangerous to scuba divers because they prevent equalisation of pressure on either side of the ear drum. Hence a need exists for a device for protecting human ears which are comfortable, unobtrusive and easy to use, cheap to produce and which effectively protect the user's ears from damaging sounds, optionally whilst leaving some sounds audible.

Phobic behaviour in response to sounds such as fireworks is exhibited by large numbers of domestic animals. Phobic behaviours caused by a hypersensitivity to sounds includes by way of example only: barking for extended periods in dogs triggered by noise; dogs who's travel stress is triggered and maintained by certain noises; and extreme fear in horses, dogs and cats in response to certain sounds especially loud and unpredictable sounds such as those produced by fireworks.

Currently there is no treatment for this distressing condition even though it affects large numbers of pets and their owners. A need exists for a means of treating hypersensitivity to sounds in sound phobic animals.

The present invention provides a safe and reversible treatment for sound phobic animals comprising an acoustic dampening fluid composition in the form of ear drops, gels, foams, pellets, liquids, emulsions, foams or like compositions which “dampen” hearing sufficiently to reduce sound hypersensitivity symptoms in sound phobic animals.

In a first aspect the invention relates to an acoustic dampening fluid composition for temporarily, reversibly impairing hearing in animals or humans comprising: an oily or non-aqueous substance and an inert solvent.

Preferred features of the composition are defined in claims 2 to 9.

In a second aspect the invention relates to a method of temporarily impairing hearing by applying an acoustic dampening fluid to the external ear canal of an animal or human.

Preferred features of the composition are defined in claims 11 and 12

In another aspect the invention relates to a method for desensitising animals to sounds comprising the following steps:
a) applying a first acoustic dampening fluid to the ears of an animal;
b) exposing the animal to the offensive sound or sounds until some tolerance to the sound or sounds has been established;
c) allowing or causing the first acoustic dampening fluid to be dispersed from the ears;
d) applying a second acoustic dampening fluid having decreasing acoustic absorptive and/or dampening properties to the ears;
e) and repeating the exposure to the offensive sounds until tolerance to the sound has been re-established.

### DETAILED DESCRIPTION

According to the present invention an acoustic dampening fluid composition is applied to the external ear canal to temporarily impair hearing and reduce sensitivity to sound.

The acoustic dampening fluid composition is applied to the external ear canal. The composition fills both the external ear canal and covers the ear drum. This both dampens vibration of the ear drum and absorbs sound waves as they enter the ear canal.

The acoustic dampening fluid composition is inert, and non-irritant. Preferably the acoustic dampening fluid composition comprises an oily or non-aqueous component.

Preferably the oily or non-aqueous substance is present in an amount of 1.0-30% m/m [what units are these please as they need to be SI units?], more preferably 5.0-20% m/m.

Preferably the acoustic dampening fluid composition further comprises a suitable solvent such as by way of example water, lower alcohols, glycols and combinations thereof.

Preferably the solvent is present in amounts of 10-95% m/m, more preferably 40-80% m/m.

Preferably the dampening fluid compositions according to the present invention are provided as a metered aerosol, a single-shot vial, or as a two-compartmental system such as a “bag-in-can” type aerosol product. Packaging in a pressurized container is desirable to mitigate against premature expansion of the mousse. However the mechanism which delivers the foam or mousse into the external ear canal must be silent so as not to alarm or distress the animal or human.

Preferably the acoustic dampening fluid remains in the ear for a short period ranging from between a few hours and a few days where it causes a temporary period of impaired hearing. More preferably the acoustic dampening fluid remains in the ear for 3 hours before beginning to disperse.

Thereafter the acoustic dampening fluid degrades and/or disperses from the ear drum and ear canal returning the hearing to its former normal level of sensitivity.

In the unlikely event of an adverse or allergic reaction being provoked by the method of the present invention the liquid, emulsion, or foam can be rapidly dispersed using an inert, non-irritant solvent.

Preferably the acoustic dampening fluid composition further comprises one or more of: a smart liquid or gel, a smart emulsion, an expanding foam and a non-expanding foam. Smart materials as used herein are materials which have one or more properties that can be significantly changed in a controlled manner in response to external stimuli. By way of example only post-foaming compositions are liquids or gels in their packaging which alter their state and become for example a foam or mousse once exposed to any of air, moisture, release from a pressurised container or mixing of multiple components together.

Optionally the acoustic dampening fluid composition further comprises one or more thickeners.

Especially preferred thickeners may be selected from: povidone, cellulose gums such as sodium carmellose, xanthan gum and other pharmaceutically acceptable thickeners, or combinations thereof.

These thickeners may also act as stabilisers for the composition and help it slowly develop from a gel or liquid to a foam or mousse after application to the external ear canal, as well as improving the mucoadhesive and skin adhesive qualities of the formulation.

Preferably thickeners are present in an amount of 0.1-30% m/m, more preferably 1.0-15% m/m.

Optionally the acoustic dampening fluid composition may further comprise one or more surfactants which assist in the formation of the foam or mousse, the emulsification of the vapour pressure reducers, and the solubilisation of the active agents. Especially preferred surfactants may be selected from nonoxinol, polysorbate, glyceryl stearate, glycol stearate, sorbitan esters, polyoxylated stearates, polyoxylated castor oil, macrogol alkyl ethers such as macrogol lauryl ether, and other pharmaceutically acceptable surfactants or combinations thereof.

Preferably surfactants are present in an amount of 1.0-60% m/m, more preferably 4.0-40% m/m.

Optionally the acoustic dampening fluid composition may further comprises one or more vapour pressure reducers. Vapour pressure reducers are often oily substances such as for example caprylic/capric triglycerides. They may act as solvents or be used in conjunction with foaming agents.

Optionally the acoustic dampening fluid composition may further comprises one or more excipient such as by way of example only pH buffers, solvents, carriers, diluents and preservatives. Suitable pH buffers may include citrates, phosphates or acetates, or other pharmaceutically acceptable buffers. Suitable preservatives may include benzyl alcohol or parabens, and other pharmaceutically acceptable preservatives. Suitable carriers and diluents may include purified water.

Preferably pH buffers are present in an amount of 0-10% m/m, more preferably 0.1-2.0% m/m.

Preferably preservatives are present in an amount of 0.01-5.0% m/m, more preferably 0.2-2.0% m/m.

By adjusting the various constituents of the dampening fluid composition the skilled addressee can adjust a number of its characteristics for example:
viscosity
adhesion to skin of the external ear canal
duration of persistence of the foam or mousse
nature of the spent foam or exhausted components
how easy spent foam is removed from the external ear canal by the body's natural mechanisms
if a “cleaner” is required to remove the spent foam.

Dampening fluid compositions according to the invention may be prepared by blending all components into the preferred solvent using an overhead stirrer.

Alternative mixing protocols are also envisaged such as by way of example only using two cylinders with pistons connected to receiving compartments for the various components of the dampening fluid. The pistons share a shaft and cylinders connect via a conduit. As the pistons cycle the components of the compound are mixed.

In preferred embodiment the acoustic dampening fluid composition is provided as a post foaming composition.

In an especially preferred embodiment the acoustic dampening fluid composition is provided as liquid or gel at room temperature which is easily administered to the ear canal.

Shortly thereafter as a result of the inclusion of foaming agents in the composition the fluid slowly foams at a rate which neither alarms or distresses the animal or human to a degree sufficient to adequately coat the skin surface within the external ear canal so as to provide a physical barrier to noise.

Foaming agents are pharmaceutically acceptable propellants. Suitable foaming agents may be selected from but are not limited to hydrocarbons such as n-butane, chlorofluorocarbons, hydrofluorocarbons, dimethyl ether, nitrogen, nitrous oxide, oxygen, ozone and carbon dioxide.

The acoustic dampening fluid composition must be formulated so that the foaming agent does not cause excessively rapid expansion of the foam in the external ear canal of an animal or human since discomfort and/or pain will result.

Once the foam collapses its spent components are small in volume and are quickly and easily cleared by the ears natural mechanisms to effect dispersal of the acoustic dampening fluid. If necessary, or desired a solvent or cleaner can be applied to accelerate removal of the spent foam.

The safety, ease of use and reversibility of the method of the present invention makes it ideally suited to the treatment of sound phobias in animals or providing a convenient means of protecting human hearing in individuals exposed to damaging noise levels for short periods eg those operating machinery or being exposed to loud music.

The acoustic dampening fluid in the form of ear drops, gels, foams, pellets, liquids, emulsions, foams or like compositions provide an alternative to conventional ear plugs and ear protectors which are impractical in animals and [disadvantages of ear defenders for humans] cumbersome and unsightly for humans.

The invention also relates to a method of progressively desensitising animals to sounds to which they are hypersensitive. The method involves applying a first acoustic dampening fluid composition to the ears of an affected animal and exposing the animal to the offensive sound or sounds. Then once some tolerance to the sound or sounds has been established dispersing the first acoustic dampening fluid composition from the ears and applying a second acoustic dampening fluid composition having decreased acoustic absorptive/dampening properties to the ears and repeating the exposure to the offensive sounds until tolerance to the sound has been re-established. This is a classical desensitisation protocol for desensitising individuals to a stimulus to which they have a phobia.

Preferably the second acoustic dampening fluid composition is obtained by diluting the first acoustic dampening fluid with a suitable solvent.

1-13. (canceled)

14. An ear defender comprising an inert acoustic dampening fluid composition which in use temporarily, reversibly impairs hearing in animals or humans comprising: an oily or non-aqueous substance and an inert solvent.

15. An ear defender according to claim 14 wherein the oily or non-aqueous substance comprises 10-30 g/kg of the acoustic dampening fluid composition.

16. An ear defender according to claim 14 wherein the oily or non-aqueous substance comprises 50-200 g/kg of the acoustic dampening fluid composition.

17. An ear defender according to claim 14 wherein the solvent is selected from the group consisting of water, lower alcohols, glycols and combinations thereof.

18. An ear defender according to claim 14 wherein the solvent comprises 100-950 g/kg of the acoustic dampening fluid composition.

19. An ear defender according to claim 14 wherein the solvent comprises 400-800 g/kg of the acoustic dampening fluid composition.

20. An ear defender according to claim 14 further comprising one or more of: a thickener, a surfactant, a smart liquid, a smart gel, a smart emulsion, foaming agents and vapour pressure reducers.

21. An ear defender according to claim 14 further comprising one or more excipient, pH buffers, solvents, carriers, diluents and preservatives.

22. A method of temporarily impairing hearing by applying an acoustic dampening fluid to the external ear canal of an animal or human.

23. A method according to claim 22 wherein the animal is a cat, a dog, a horse, a rabbit or like domestic animal.

24. A method for desensitizing an animal to sounds comprising the following steps:
applying a first acoustic dampening fluid to at least one ear of an animal;
exposing the animal to the offensive sound or sounds until some tolerance to the sound or sounds has been established;
allowing or causing the first acoustic dampening fluid to be dispersed from the ear;
applying a second acoustic dampening fluid having decreasing acoustic absorptive and/or dampening properties to at least one ear of the animal; and
repeating the exposure to the offensive sounds until tolerance to the sound has been established.
